(19) United States
(12) Patent Application Publication
(10) Pub. No.: US 2002/0156345 A1
(43) Pub. Date: Oct. 24, 2002

(54) Method of guiding an endoscope for performing minimally invasive surgery

(76) Inventors: Wolfgang Eppler, Karlsruhe (DE); Ralf Mikut, Karlsrhe (DE); Udo Voges, Stutensee (DE); Rainer Stotzka, Karlsruhe (DE); Helmut Breitwieser, Muggensturm (DE); Reinhold Oberle, Bretten (DE); Harald Fischer, Karlsruhe (DE)

Correspondence Address: KLAUS J. BACH & ASSOCIATES, PATENTS AND TRADEMARKS, 4407 TWIN OAKS LANE, MURRYSVILLE, PA, 15668, US

(21) Appl. No.: 10/172,436

(22) Filed: May 16, 2002

Related U.S. Application Data
(63) Continuation of application No. PCT/EP00/11062, filed on Nov. 9, 2000.

(30) Foreign Application Priority Data
Dec. 22, 1999 (DE) 199 61 971 . 9

(51) Int. Cl. A61B 1/01

(52) U.S. Cl. 600/114; 600/118; 600/102

(57) ABSTRACT

In a method of guiding an endoscope for performing minimally invasive surgery, wherein a surgical instrument is automatically tracked by an electrically driven and controlled guide system (EGS), three base steps are principally followed: the computer controlled processing of fault tolerances, the intuitive use of the equipment by the surgeon and the sovereignty of the operating surgeon. In this way, a high degree of reliability during operation is achieved and the surgeon is relieved from the tasks of performing the tracking procedures which requires a high level of concentration and from carrying out tasks of relatively low priority.

This is a Continuation-In-Part application of international application PCT/EP00/11062 filed Nov. 9, 2000, and claiming the priority of German application 199 61 971.9 filed Dec. 22, 1999.

### BACKGROUND OF THE INVENTION

The invention relates to a method for safely and automatically guiding an endoscope and for tracking a surgical instrument, including an electrically operated and controlled endoscope guide system (EGS) for minimally invasive surgery.

During minimally invasive surgery, the surgeon orients himself on the basis of a monitor (original monitor). An endoscope including a camera and the instruments needed for the surgery are inserted into a body cavity through a trocar.

Presently, the endoscope as well as the camera are moved generally manually: The surgeon who controls the instruments advises an assistant to follow the movement of the instrument with the endoscope and the camera so that the instrument remains visible on the monitor screen. The advantage of this procedure is that the assistant guiding the endoscope avoids dangerous situations, recognizes errors, communicates with the surgeon and follows with the endoscope only when this is necessary. The disadvantage is the need for additional personnel in comparison with conventional surgery and the unavoidable jittery movement of the assistant.

In order to avoid the above-mentioned disadvantages systems have been introduced which guide the endoscope automatically. Such endoscope guide systems for guiding an endoscope camera unit is electrically operated and can be mounted on any surgery table. For the remote operation, it includes an operating component, generally a joystick, which is generally connected to the operating instrument or it may be provided with a speech input. The endoscope inserted into a body as well as separately inserted instruments have generally each a fixed point with respect to their movement, which is the trocar penetration point which must be in, or on, the body wall of the patient so that the apparatus can be pivoted and tilted without injuring the patient more than he or she has already been injured by the penetration with the trocar. The camera of the endoscopic system is then so guided and mounted that the lower image edge extends parallel to the patient support and the image is not upside down (see for example DE 196 09 034). A rotation of the camera is possible, but it makes the spatial orientation more difficult.

An endoscope of such an endoscope guide system, which extends into the body of a patient, has several degrees of freedom. The EGS described in DE 196 09 034, for example, has four degrees of freedom of movement: It can be tilted about a first axis extending normal to the surgery table and through the body penetration point, about a second axis extending normal to the first axis and normal to the penetration direction, it can be moved along a third axis, the trocar axis, and it can be rotated about the trocar axis. Movements in the first three degrees of freedom are limited by limit switches. With an operating component disposed for example at the instrument handle of the instrument operated by the surgeon, the endoscopic camera is for example controlled as to its viewing direction.

In each of the four degrees of freedom, the instruments can be adjusted with a speed which is limited for safety reasons.

For an endoscopic control system operating on the basis just described an automatic tracking system is provided. Such a control system is known from U.S. Pat. No. 5,820,545. The respective instrument tip is adjusted herein so as to follow constantly each movement, which results in a commotion for the observer. It also requires a special electronic control system, which is quite involved and expensive. If the third dimension is to be covered, a special 3D camera must be provided which complicates the equipment and makes it more expensive. An error adjustment as it may be necessary because of reflections or varying illuminations is not provided.

In the tracking system according to U.S. Pat. No. 5,836,869, the image tracks the instrument tip. The operating surgeon can see two different images. Color geometry or light coding of the instrument and position recognition by way of magnetic probes at the operating instrument are described. Two images can be observed, that is, the zoom image of a particular area and an overview. The tracking is based on the instrument or on color—or location—marked organs. Multicolor markings for switching the tracking target and for increasing the safety by redundancy are mentioned. The control member is in each case the camera zoom or, respectively, the position of the CCD-chip in the camera or an electronically obtained image selection on the monitor. The system uses special cameras throughout.

In all used methods, there are more degrees of freedom available than are necessary for the positioning of the EGS, in order to bring the instrument tip to the desired position. The degrees of freedom are used to minimize the amount of movement to be performed. A possible method is the determination of optimal control values utilizing a Jacobs matrix, wherein also control restrictions may be included (U.S. Pat. No. 5,887,121).

None of these methods has the advantages obtained with the manual guiding by an assistant. The tracking is furthermore still jittery since the systems try to accurately reach a certain point on the monitor by tracking even small movements the instrument with the endoscope. Furthermore, the systems are not in a good position to automatically detect errors. There is only a simple unidirectional communication from the surgeon to the EGS. The surgeon obtains no hints concerning possible sources of errors.

It is the object of the present invention to provide a fast error-tolerant and inexpensive method for automatically tracking an instrument tip with an endoscope which is carefully moved so as to eliminate for the surgeon the need of guiding the endoscope during surgery.

### SUMMARY OF THE INVENTION

In a method of guiding an endoscope for performing minimally invasive surgery, an endoscope wherein a surgical instrument is automatically tracked by an electrically driven and controlled guide system (EGS), three base steps are principally followed: the computer controlled processing of fault tolerances, the intuitive use of the equipment by the surgeon and the sovereignty of the operating surgeon. In this way, a high degree of reliably during operation is achieved and the surgeon is relieved from the tasks of performing the tracking procedures, which requires a high level of concentration and also from carrying out tasks of relatively low priority.

With the method according to the invention, the advantages of a manual guiding of the endoscope are maintained for the automatic tracking.

The safety concept on which the method is based includes several stages:

A. Error tolerance handling

B. The intuitive operation and

C. Sovereignty.

The image processing and endoscopic control part is strictly separated from the base-monitor of the operating surgeon. Errors in these parts affect not only the sequences followed thereby. The recognition of the instrument tip and the control of the endoscope with its axes and the zoom control are treated as a unit since the safety concept provided therewith can determine errors in the image recognition and also in the setting of the control value with high reliability. Error conditions that can be determined are:

Multiple image recognition of the instrument because of reflections, no image recognition of the instrument because of soiling, time-delayed recognition of the instrument to such an extent that the scanning rate of the endoscope control cannot be maintained because of insufficient computer power, Unrealistic sudden location changes of the instrument because of a limited speed of the control motors and an excessive safety-critical approximation of the lens to the instrument or an organ.

The endoscopy adjustment is only changed when the instrument tip leaves a certain frame in the center of the image of the monitor (reliable range). In this way, the picture for the surgeon remains unchanged as long as he moves the instrument within this frame in the center area of the image.

The instrument tip is marked by its form, by color or only by its characteristic shape in order to facilitate rapid recognition thereof. Still it is unavoidable that the features change with different instruments. Therefore, an online adaptation of the characteristic properties of the marks with neural or statistic learning procedure will result in a safe and flexible instrument recognition.

For performing all these method steps standard components such as a computer and operating systems and cameras are sufficient. For the observation, a single camera, that is, a 2-D camera is sufficient. The system performs the tracking on the basis of two-dimensional image information. With the use of a 3-D camera, the use of a video channel is sufficient whereby the hardware expenses for the image processing is reduced.

The instrument tip is to be held in the center of the image of the 0 monitor. Therefore, movements normal to the image plane are not taken into consideration. If they are to be taken into consideration, for example, for a zoom control or for a camera movement normal to the image plane additional measures must be taken. One such measure is the provision of an additional sensor on the trocar of the instrument, which determines the insertion depth. In this way, the need for a two channel image processing as it is needed for a 3-D image is reduced to a single channel are ordering to 2-D images. Another possibility is to roughly calculate the distance between the endoscope and the instrument tip from the perspective distortions of the parallel edges of the instrument. This requires that the focal length of the camera as well as the width and length dimensions of the instrument are known.

Highest priorities have the actions of the operating surgeon who can interfere with the endoscope control at any time and can interrupt the tracking. Before a surgery, the equipment is adjusted during a functional examination wherein the concentric setting of the monitor ranges is set. There are three ranges on the monitor: the whole monitor area, the area in which the instruments are to be shown and the center area. The endoscope setting is automatically changed only when the instrument tip leaves the admissible area, whereby the image remains still. In order to be able to do this, the area of the instrument tip is depicted in the computer, and a model thereof sufficient for its identification is recorded. This may be done, for example, by generating a gradient image, segmenting the edges of the object and determining the third dimension by calculating the straight edge lines by means of linear regression. The gradient image may be generated, for example by a Sobel-filter.

In order to achieve a high safety quality sufficient redundancy is to be provided. The basis generation of the multi-sensor surrounding by position sensors and image processing may be supplemented by additional position sensors on the guide system of the instrument or by determining the insertion depth of the trocar.

The advantage of redundancy resides in the fact that the image processing and the redundant sensors have different advantages and disadvantages. For example, the image processing is sensitive to a cover-up of the instrument tip and soiling of the lens. Position sensors at the instrument guide system may supply incorrect information—depending on the measuring principle used—; if there are electromagnetic disturbances in the operating room; they may be inaccurate because of different lengths of different instruments; or inaccuracies in the determination of the reference coordinate system between the endoscope and the instrument guide system; or the instruments may fail during surgery. If there are image processing as well as position sensors for the guidance of the instrument, the results may be compared and examined for consistency. Based on the development of the errors in many cases, conclusion can be drawn as to which of the sensor signals represent the current situation without error.

The use of the position sensors at the instrument shaft or at the instrument guide system may even permit total replacement of the image processing.

The degree of redundancy of the degrees of freedom of movement of the endoscope guide system is determined by the number of excess axes which are not directly necessary for the centering of the object in the 0-monitor image. These may be extra-corporal axes of the EGS-rotation about a vertical axis, about a horizontal axis and rotation about, as well as, translation along, the trocar axis. There may be further degrees of freedom, which may result from the use of endoscopes with flexible pivotable distance ranges. In this way, there are even so-called intra-corporal axes or respectively degrees of freedom.

This concept provides for a high degree of safety and a high error tolerance. The method operates, in simple recognition situations, with a relatively high processing speed particularly during image processing and is in a position under complicated recognition conditions, such as unfavorable illumination and similarities between the instrument tip and the ambient area, to track with a reduced speed. However, tracking of the endoscope is always fast enough so as not to provoke the impatience of the surgeon.

Since the endoscope is subjected by the guide system to only relatively little movement, there is on the monitor a relatively still, yet time passage which does not unnecessarily distract the surgeon, which facilitates the surgeons task.

The method permits an optimal integration of additional sensor information such as magnetic sensors at the guide system of the operating instrument, measurement of the insertion depth at the trocar to compensate in a multi-sensor environment for the temporary failure of individual sensors by soiling of the instrument tip with optical measurement procedures, to examine the likelihood of the sensor information obtained and, as a result, to improve the safety.

If the instrument is guided by an Instrument Guide System either by hand or by a machine, information is supplied also in this way to the IGS.

The system is composed of commercially available components as partial systems and can therefore be realized in an economic manner.

The system will be explained in greater detail on the basis of the accompanying drawings.

### BRIEF DESCRIPTION OF THE DRAWINGS

FIG. 1 shows the hierarchy of the method according to the invention,

FIG. 2 shows the system structure,

FIG. 3 shows various operating states during automatic tracking,

FIG. 4 shows the image areas on the monitor,

FIG. 5 is a representation of the instrument geometry, and

FIG. 6 shows schematically the endoscopic system.

### SYSTEM DESCRIPTION

In medical apparatus, the safety standards are very high. The core of the automatic endoscope tracking is therefore the error-tolerant method, which operates with multiple redundancy and therefore ensures the required safety. Additional safety occurs with the relief of the operating surgeon who is freed from technical procedures. Various degrees of automatic tracking support and the surgeon as he or she desires. As a result, the surgeon can operate the instruments necessary for an operation intuitively and in a sovereign manner. This is ensured by the still guidance by the speed limit during tracking and the voice system by which the surgeon is kept informed by way of: MMI-Monitor, LCD display or voice information concerning errors and critical conditions of the system such as soiling of the endoscope.

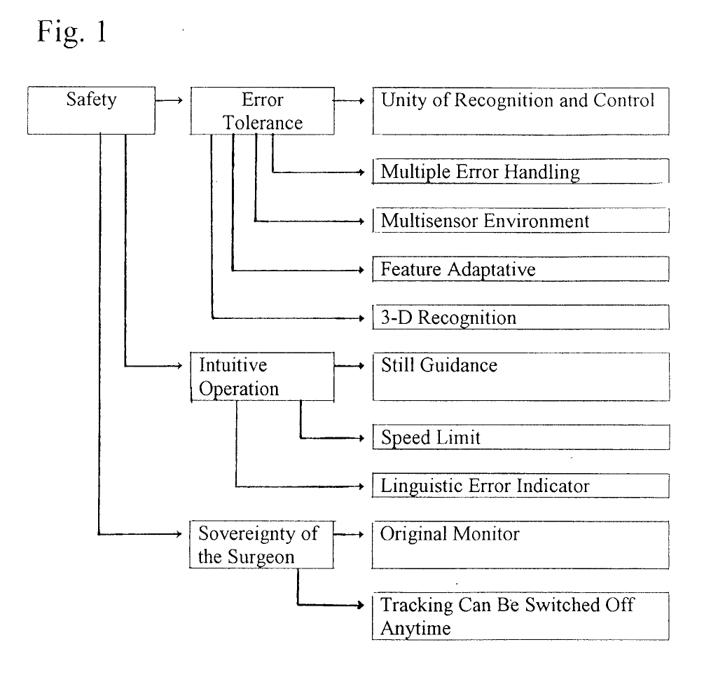

In this way, the safety and acceptance is, in comparison with presently available systems, substantially improved because the surgeon or an assistant can eliminate the causes for malfunction effectively and rapidly for example by cleaning the optical system or by returning the instrument to the proper image area. In addition, unexpected reactions of the tracking system are substantially reduced. Sovereignty further means: The surgeon uses the monitor which does not depend on the tracking systems, that is, the original monitor and has the hierarchic possibility to switch off the tracking system at any time. FIG. 1 shows this structured requirement and it also shows the hierarchy structure starting with the central requirement for safety.

The error tolerance is achieved by one or more measures:

Object recognition and control as a unity, multiple treatment of possible error conditions resulting from individual components of the image processor and the control as well as a superior surveillance unit.

Multi-sensor concept,

adaptive feature adaptation, and

3-D reconstruction.

The advantage of the uniform treatment of the object recognition and control resides in the fact that the causes for errors can be pinpointed. If, for example, the last setting actions are known the likely positions of the instrument markings can be assumed with relatively high accuracy, whereby an improved recognition safety can be achieved. A determination of the reasons for errors has, in addition to an improved communication with the surgeon, the advantage that adequate system reactions can be determined.

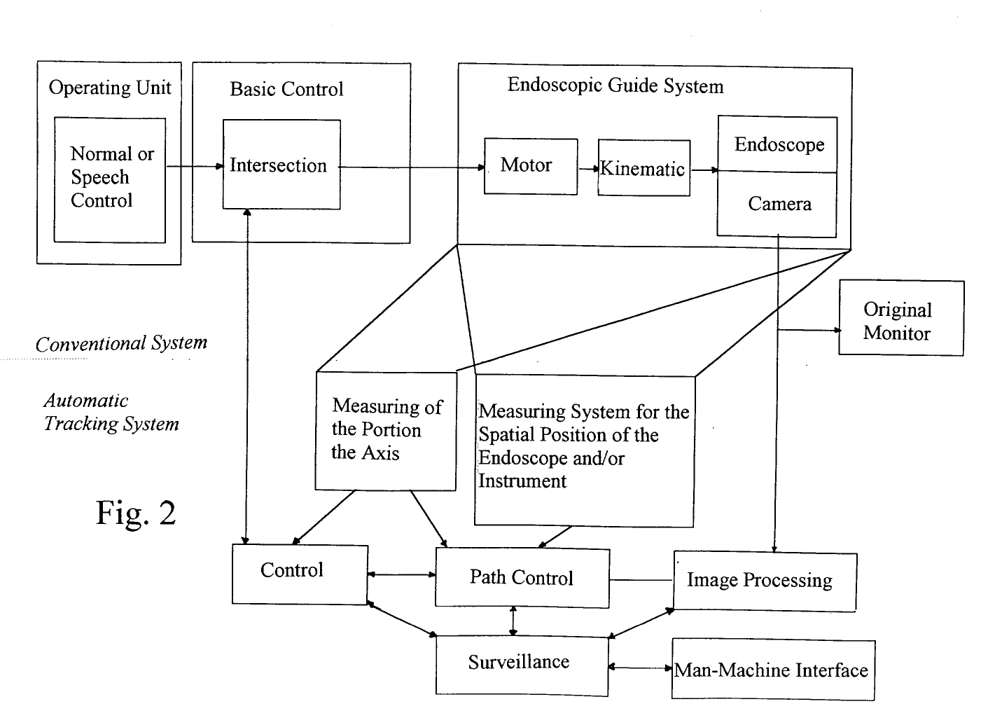

A system configuration of the endoscope guide system is schematically presented for example by the system structure of FIG. 2 and comprises the following blocks which are interconnected by a cable,

the basic EGS with four degrees of freedom, left/right, top/bottom, rotating and in/out including the electronic control and the limit switches on the respective axes of the degrees of freedom,

the 2-D video endoscope with video output (red/yellow/blue-output, RYB), original monitor and light source,

the computer (PC) with MMI monitor for the Man-Machine Interface (MMI) and the digital output card for the control of the logic interface (TTL),

the additional components for the image processing, so-called frame grabber,

the operation interface in the form of a manual switch, the joystick, for the manual operation.

The tracking control consists of the following components:

Image processing,

Track control and,

Surveillance.

It processes the input values:

B1=Binary Input “Tracking in”,

B1=Binary Input “Tracking stop”, and

The video signal with three channels (RGB) and synchronization.

The output values are:

2×4×BO (Binary Output) for changing the positions of the axes by addressing a second digital interface,

status and error messages.

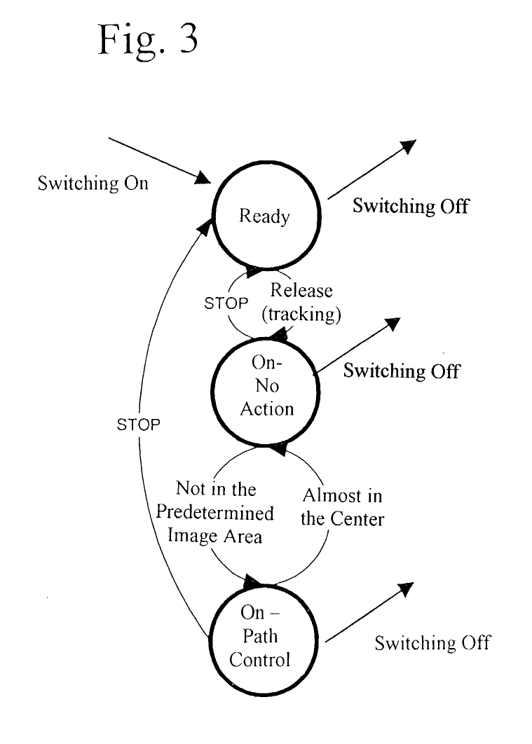

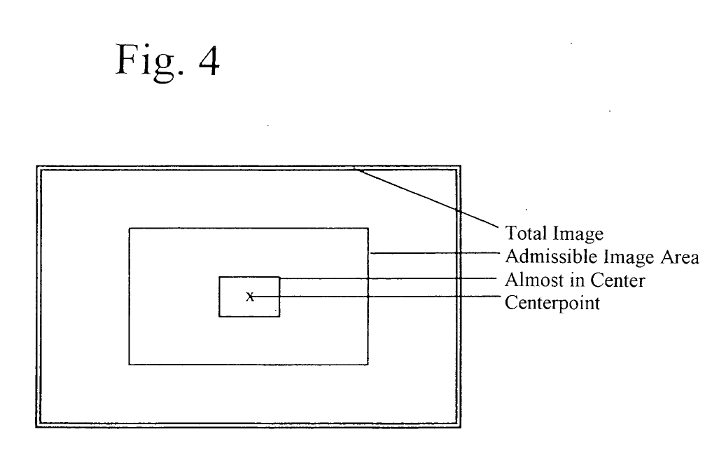

The main object of the automatic tracking function resides in the fact that the momentarily needed instrument tip is to be maintained in the center area of the monitor (see FIG. 4). The control procedure required therefor is presented in the condition graph of FIG. 3. The release switching for the automatic tracking is initiated within the system.

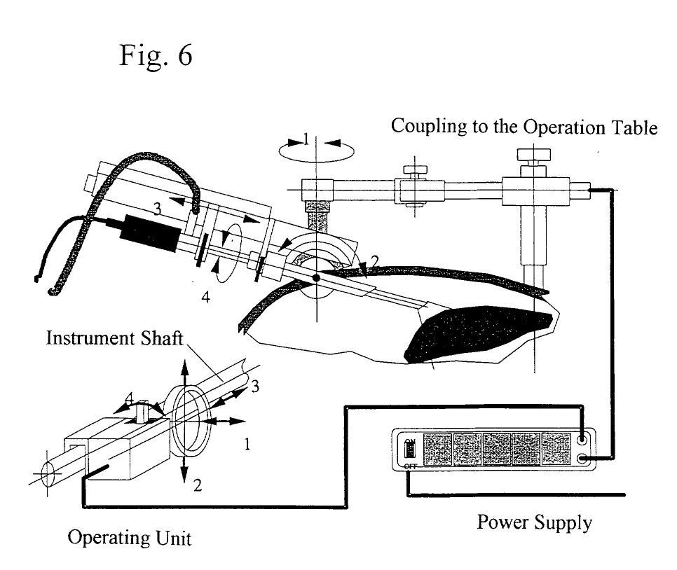

The automatic tracking is initiated in the present case by the operating surgeon by way of the ring switch at the operating unit (see FIG. 6) It remains activated until it is stopped either by pressing the stop button or by joystick actuation or automatically.

The tracking is automatically stopped,

when no instrument is recognized within the image either because none is present or because of soiling of the system,

when the image becomes blurry because the instrument is too close to the camera,

when the instrument cannot be recognized within the required reaction time,

when no video signal is present,

when the image processing, the tracking control the surveillance or the control recognizes electronic or program errors. Any errors are indicated on the MMT monitor.

After a stop, the tracking can again be initiated. The automatic tracking operates with predetermined limited adjustment speeds up to 10 cm/see or respectively, 30°/sec, which can be adjusted depending on applications (belly; lungs; heart surgery for example) in an individual-dependent manner in such a way that the surgeon can react to undesired situations. Furthermore, there is a control limit for the positions of the axes, which keeps tilting and pivoting within predetermined limits, which limits the translatory movement along the trocar axis and which does not permit a full rotation about the shaft axis (see FIG. 7).

From the camera image of the O-monitor (see FIG. 4), the possibly additionally marked instrument tip is automatically recognized by comparison with an image thereof stored in the computer and its average position by the x and y locations in the two-dimensional camera image, recognition probability, size of the identified instrument tip and additional information for error recognition are supplied to the control. The recognition of the instrument tip operates automatically and is independent on the tracking release. The image processing (FIG. 2) recognizes any errors such as no instrument in the image frame, several instruments in the image frame, and stops the automatic tracking in such cases. When the instrument tip leaves the admissible image area (FIG. 4) the automatic tracking system will change the position of the camera or the endoscope such that instrument tip is again in the center area of the image. This task is solved by the track control (see FIG. 2), which continuously processes the measured position of the instrument tip in the camera image.

When the instrument tip is again within the smaller area (almost in the center—FIG. 4) around the center of the image, no position adjustment is initiated until the instrument tip again leaves the larger admissible area in the image. With this reservation in the movement by area-dependent suppression of the tracking movement a still picture is generated on the O-monitor.

The status of the automatic tracking and any error messages are displayed on the monitor, while the image is displayed so that the image transmission to the monitor is not interrupted.

In order to obtain depth recognition, generally a 3-D position determination is employed. But, since in that case, two cameras would be necessary and arranged at different observation angles, a depth recognition on the basis of 2-D image data using only one camera is preferably used. Employing the simple beam-optical relation between image and subject distances permits the determination of the distance

g=f(G/B+1)

wherein g=the distance of the object, G=the size of the object, B=image size, f=focal length of the endoscope lens.

The most important object of the depth estimation is the determination of the size of the object in the image. The “object”, may also be represented by easily recognizable markings at the sharp edges on the object. The most simple recognition method resides in the determination of the diameter of segmented marking regions. However, this has been found to be inaccurate since, with different orientation of the endoscope and because of the properties of the central projection, there may be deformations which do not permit an accurate determination of the width of the object.

A better method for determining the instrument width at the tip segments, in a first step, the edges of the object and then determines the distance from the calculated center point. This has the advantage that the width of the object is determined independently of the orientation of the object and unaffected by the particular projection.

The object edges can be detected in several steps:

First, a filter, for example, a 3×3 Sobel filter is applied to the transformed shading values of the image in order to subsequently begin an edge determining algorithm.

The edges determined in this way however have the disadvantage, that their width may vary substantially. However, a thin edge line is required which has the width of a pixel in order to facilitate a determination of the distances from the edge in an accurate manner.

This is achieved by replacing the segmented edges by approximated straight lines.

This is achieved fastest by a linear regression analysis, wherein the relation between the x and y values of a quantity of line point are formulated in the formulated in the form of a linear model. In this way, the edges can be mathematically defined which facilitates the determination of the size of the object in a next step.

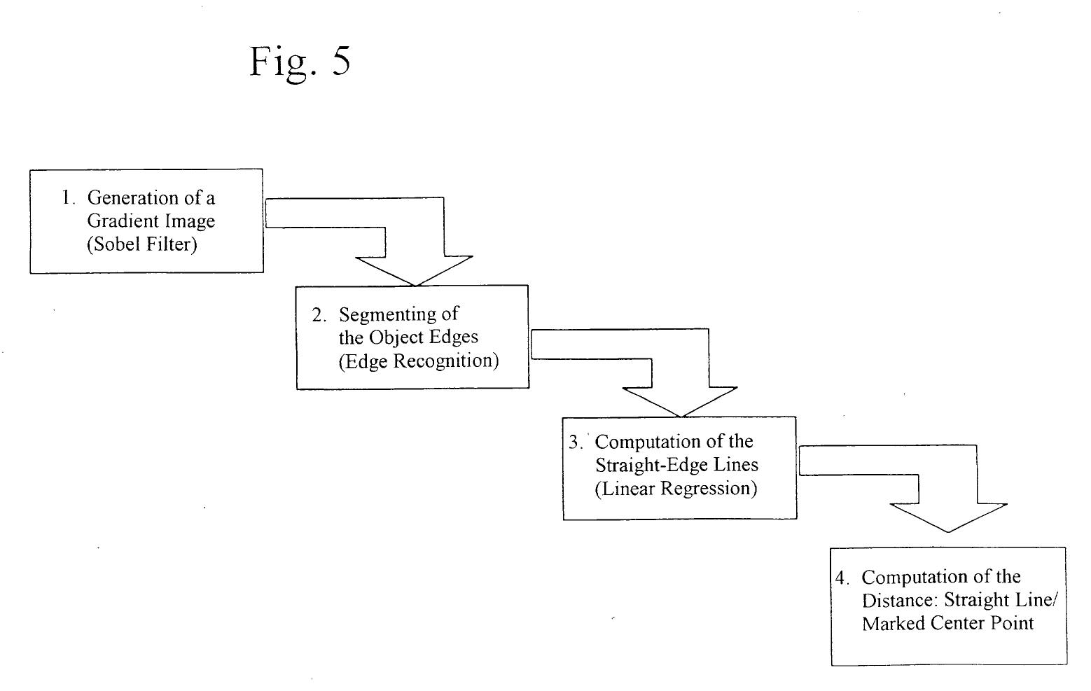

This is done either by way of the distance between two parallel straight lines or by way of the distance of a straight line from the center point of the object by transformation of the line equations into the Hesse normalized form and insertion of the center point. FIG. 5 is an overview showings the method including the four essential steps.

These are:

1. Generation of the gradient image of the marked instrument using the Sobel filter, then

2. Segmenting the edges of the object, tracking the edges, then

3. Calculating the straight edge lines by means of linear regression and finally

4. Calculating the distance: Straight line—center point of markings.

It is noted that the accuracy of the distance determination depends essentially on the quality of the edge extraction.

What is claimed is:

1. A method for safely automatically guiding an endoscope and for tracking a surgical instrument with an electrically operated and controlled Endoscope Guide System (EGS) for performing minimally invasive surgery, said method comprising the following steps:
Error tolerance processing:
Taking a photograph of the distal end area of an instrument used in the surgery and storing a specific copy thereof with actual position values in an image processing system, observing the instrument and recording as an error the occurrences of multiple recognition because of reflections, no recognition because the object is not within an image frame, no recognition because of cover-up, no recognition because the image is not sharp as a result of an insufficient distance between the lens and the instrument tip, time-delayed recognition because of insufficient computer power and sudden location changes as a result speed limit of control motors.
discontinuing tracking of the EGS upon recognition of critical errors in order to avoid injuries to a patient,
with the use of a camera with image processing and position sensors for the degrees of freedom of the EGS, generating a multi-sensor environment, wherein the endoscope guide system compensates for the temporary failure or the ineffectiveness of individual sensors under certain operating conditions such as covering of the instrument, soiling of the lens, electromagnetic disturbances, and examines the actually evaluated sensor information for reasonability, performing a recognition procedure by adaptive feature adaptation for recognizing different objects by machine, neural or statistical learning procedures,
treating possible error states at least partially twice, specifically by individual components of the image processing and the movement control and by a supervisory control-based surveillance unit,
calculating from the perspective distortion of parallel edges in the distal instrument area the distance between the observing endoscope and the instrument tip taking into consideration the focal length of the camera lens and the sized of the instrument (3-D reconstruction);
Intuitive Operation
changing the position of the endoscopic only when the instrument tip visible on the original monitor (0-monitor) leaves a predetermined central area (admissible area), whereby a still image is obtained as no unnecessary adjustment movement are executed,
indicating the cause for an error detected in case of error by way of a Man-Machine Interface (MMI), which consists of at least one of MMI monitor and a speech output so as to facilitate active measures by the surgeon for the elimination of the error such as cleaning of the camera lens or manually returning the instrument tip into the image frame.
Sovereignty
the actions of the operating surgeon and observed by him on the O-monitor have priority and are not influenced by the endoscope guide system:
the endoscope guide system with its error tolerance processing and intuitive operation is switched on by the surgeon when needed and switched off when not needed;
the speed of the tracking of the instrument and the angular speed of rotating the instrument is so limited that the surgeon can interfere upon incorrect processing in complicated recognition situations such as unfavorable illumination and similarities between instrument tip and surroundings.

2. A method according to claim 1, wherein the image of the O-monitor is divided during a functional examination for the automatic tracking which precedes an operation, into three differently sized concentric areas:
a center area: if the instrument or instruments are in the center area the endoscope is not tracking,
an admissible area extending around the center area: if the instrument or instruments are within this area the endoscope is automatically tracking if the instrument or instruments had left the area previously and,
an outer area extending around the admissible area: if the instrument or instruments are disposed in this area the endoscope automatically is tracking with the arm to return the instrument to the center area.

3. A method according to claim 2, wherein the image of the instrument tip stored in the computer is a simplified model of the instrument tip.

4. A method according to claim 3, wherein, of the area of the instrument tip, which may be specifically marked, first a gradient image is generated, the object edges are segmented by tracking the edges and the respective straight edge line is calculated by linear regression in order to determine therefrom the third dimension.

5. A method according to claim 4, wherein the gradient image is generated by means of a Sobel filter.

6. A method according to claim 5, wherein the multi-sensor environment generated by the position sensors is complemented by position sensors at the guide system of the surgical instrument whereby failures in one system are compensated for by values generated in others.

7. A method according to claim 5, wherein the multi-sensor environment generated by the camera with image processing and the position sensors is complemented by measuring the insertion depth at the trocar, whereby failures in one system are compensated for by values generated in others.

8. A method according to claim 5, wherein the redundancies generated by the extra-corporal degrees of freedom of the EGS are expanded for the tracking by the intra-corporal degrees of freedom of the EGS.

9. A method according to claim 8, wherein, for tracking the area of the instrument tip, a 2-D camera or a 3-D camera of which only one image channel is used for the image processing is utilized for reducing the hardware expenses.
